(19) United States
(12) Patent Application Publication
(10) Pub. No.: US 2007/0239165 A1
(43) Pub. Date: Oct. 11, 2007

(54) Device and method of spacer and trial design during joint arthroplasty

(76) Inventor: Farid Amirouche, Highland, IL (US)

Correspondence Address: BRINKS HOFER GILSON & LIONE, P.O. BOX 10395, CHICAGO, IL, 60610, US

(21) Appl. No.: 11/394,306

(22) Filed: Mar. 29, 2006

(51) Int. Cl. A61F 5/00; A61F 2/30; A61B 5/103

(52) U.S. Cl. 606/86; 623/18.11; 600/587

(57) ABSTRACT

A spacer block for gathering data to be used in selection of a trial insert includes a first body piece, a second body piece positioned on top of the first body piece, and at least one chim positioned on top of the second body piece. The first body piece includes at least one sensor to measure forces between the first and second body pieces, and the spacer block includes a processor having a memory operatively coupled to the sensor. The data can be analyzed using a trained neural network to provide feedback to a physician to aid in the determination of whether to resect additional bone, release soft tissues, and/or select sizes for a trial insert. Advantageously, increased data may be provided to a physician without the need to acquire numerous samples from a patient, and fewer sensors may be employed.

### BACKGROUND

1. Technical Field

The invention relates to joint replacement, and more particularly, to a spacer block used to provide data to assist in selecting the size of a trial implant.

2. Related Applications

This application incorporates by reference applicant's co-pending applications U.S. patent application Ser. No. ______ (Attorney Docket No. 12462/4), filed concurrently herewith and entitled “Application of Neural Networks to Prosthesis Fitting and Balancing in Joints,” and U.S. patent application Ser. No. ______ (Attorney Docket No. 12462/6), filed concurrently herewith and entitled “Force Monitoring System.”

3. Related Art

Some medical conditions may result in the degeneration of a human joint, causing a patient to consider and ultimately undergo joint replacement surgery. The long-term success of the surgery oftentimes relies upon the skill of the surgeon and may involve a long, difficult recovery process.

The materials used in a joint replacement surgery are designed to enable the joint to move like a normal joint. Various prosthetic components may be used, including metals and/or plastic components. Several metals may be used, including stainless steel, alloys of cobalt and chrome, and titanium, while the plastic components may be constructed of a durable and wear resistant polyethylene. Plastic bone cement may be used to anchor the prosthesis into the bone, however, the prosthesis may be implanted without cement when the prosthesis and the bone are designed to fit and lock together directly.

To undergo the operation, the patient is given an anesthetic while the surgeon replaces the damaged parts of the joint. For example, in knee replacement surgery, the damaged ends of the bones (i.e., the femur and the tibia) and the cartilage are replaced with metal and plastic surfaces that are shaped to restore knee movement and function. In another example, to replace a hip joint, the damaged ball (i.e., the upper end of the femur) is replaced by a metal ball attached to a metal stem fitted into the femur, and a plastic socket is implanted into the pelvis to replace the damaged socket. Although hip and knee replacements are the most common, joint replacement can be performed on other joints, including the ankle, foot, shoulder, elbow, fingers and spine.

As with all major surgical procedures, complications may occur. Some of the most common complications include thrombophlebitis, infection, and stiffness and loosening of the prosthesis. While thrombophlebitis and infection may be treated medically, stiffness and loosening of the prosthesis may require additional surgeries. One technique utilized to reduce the likelihood of stiffness and loosening relies upon the skill of the physician to align and balance the replacement joint along with ligaments and soft tissue intraoperatively, i.e., during the joint replacement operation.

During surgery, a physician may choose to insert one or more temporary components. For example, a first component known as a “spacer block” is used to help determine whether additional bone removal is necessary or to determine the size of the “trial” component to be used. The trial component then may be inserted and used for balancing the collateral ligaments, and so forth. After the trial component is used, then a permanent component is be inserted into the body. For example, during a total knee replacement procedure, a femoral or tibial spacer and/or trial may be employed to assist with the selection of appropriate permanent femoral and/or tibial prosthetic components, e.g., referred to as a tibia insert.

While temporary components such as spacers and trials serve important purposes in gathering information prior to implantation of a permanent component, one drawback associated with temporary components is that a physician may need to “try out” different spacer or trial sizes and configurations for the purpose of finding the right size and thickness, and for balancing collateral ligaments and determining an appropriate permanent prosthetic fit, which will balance the soft tissues within the body. In particular, during the early stages of a procedure, a physician may insert and remove various spacer blocks or trial components having different configurations and gather feedback, e.g., from the patient. Several rounds of spacer block and/or trial implantation and feedback may be required before an optimal component configuration is determined. However, when relying on feedback from a sedated patient, the feedback may not be accurate since it is subjectively obtained under relatively poor conditions. Thus, after surgery, relatively fast degeneration of the permanent component may result.

Some previous techniques have relied on placing sensors that are coupled to a temporary component to collect data, e.g., representative of joint contact forces and their locations. One limitation associated with available systems that use of sensors is that, while objective feedback is obtained, that feedback is limited to the number of sensors that are employed and the number of physical tests that are performed.

Therefore, it would be desirable to obtain enhanced feedback during prosthesis fitting and balancing in joints without increasing the burden imposed upon the physician or the patient. Thus, there is a need for a spacer block that will provide enhanced feedback during prosthesis fitting and balancing.

### SUMMARY

In overcoming the above limitations and other drawbacks, a spacer block is provided that includes a first body piece, a second body piece positioned on top of the first body piece. The first body piece includes at least one sensor that measures forces, such as dynamic contact forces, between the first and second body pieces. The spacer block includes a processor that includes a memory. The processor is operatively coupled to the sensor to receive data therefrom.

In one aspect, at least one chim may be positioned on top of the second body piece.

In another aspect, the sensor comprises a plurality of load cells that are operatively connected to the processor and are adapted to measure compression, tension, and bending forces between the first and second body pieces. The first body piece includes at least one load cell associated with each chim. Each load cell is positioned to measure forces between the first and second body pieces due to forces exerted on the associated chim.

In another aspect, the first body piece includes a plurality of poles extending vertically upward such that distal ends of the poles are in contact with the second body piece. The sensor comprises a plurality of strain gauges positioned on the poles. The strain gauges are operatively connected to the processor and are adapted to measure compression, tension, and bending forces between the first and second body pieces. Each pole is positioned such that the strain gauges will measure forces between the first and second body pieces due to forces exerted on the associated chim.

In still another aspect, the spacer block includes a transmitter that is operatively connected to the processor. The transmitter is adapted to transmit data from the processor to a remote receiver.

In yet another aspect, the spacer block includes a handle detachably connected to the spacer block for manipulation of the spacer block. The spacer block and the handle include features to allow an electrical connection therebetween when the handle is connected to the spacer block. The handle can include a transmitter operatively connected to the processor through the electrical connection, wherein data from the processor is transmitted to a remote receiver, when the handle is connected to the spacer block. Alternatively, the handle may include a hard wired connection to a receiver such that data from the processor can be sent to the receiver, through the handle, when the handle is connected to the spacer block.

In still another aspect, the spacer block includes a handle that is integrally formed with the spacer block. Similarly to the detachable handle, the integrally formed handle may include a transmitter operatively connected to the processor, wherein data from the processor is transmitted to a remote receiver. Alternatively, the handle may include a hard wired connection to a receiver such that data from the processor can be sent to the receiver, through the handle.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 is a plan view of a human knee having a trial insert placed therein;

FIG. 2 is an exploded view of a spacer block of the present invention, incorporating load cells as sensors;

FIG. 3 is an exploded view of a spacer block of the present invention, incorporating strain gauges as sensors;

FIG. 3A is an enlarged portion of FIG. 3, as indicated by the encircled area labeled FIG. 3A in FIG. 3;

FIG. 4 is an exploded view similar to FIG. 3 from an angle showing an underside of the second body piece;

FIG. 5 is a perspective view of a spacer block having an integrally formed handle;

FIG. 6 is an exploded view of a spacer block having a detachable handle;

FIG. 7 is an exploded view of a portion of a spacer block having a detachable handle of another embodiment;

FIG. 8 is a plan view of a human knee having a spacer block of the present invention placed between the femur and tibia;

FIG. 9 is a block diagram depicting various components of a joint prosthesis fitting and balancing system;

FIG. 10 is a schematic showing details of a neural network that may be used in conjunction with the present invention;

FIG. 11 is a schematic illustrating the input, weighting, activation and transfer function of a node of the neural network in FIG. 10;

FIG. 12 is a block diagram showing the training phase of a neural network for use in the present invention;

FIG. 13 is a block diagram depicting the use phase of a neural network for use in the present invention; and

FIGS. 14 and 15 are views of finite element models that may be used in conjunction with the present invention.

### DETAILED DESCRIPTION

The present invention is directed to a spacer block for use in prosthesis fitting and balancing in joints. It will be apparent that the device described herein below, may be applied to a variety of medical procedures, including, but not limited to, joint replacement surgeries performed on the shoulder, elbow, ankle, foot, fingers and spine.

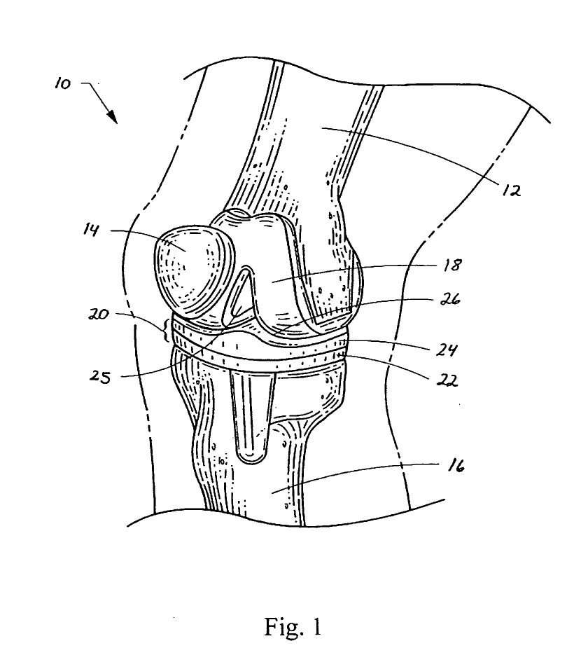

Referring now to FIG. 1, a schematic of a human knee undergoing a total knee arthroplasty (TKA) procedure is shown. In general, the human knee 10 comprises a femur 12, a patella 14, a tibia 16, a plurality of ligaments (not shown), and a plurality of muscles (not shown). An exemplary prosthesis that may be used during a TKA procedure comprises a femoral component 18 and a tibial component 20. The tibial component 20 may comprise a tibial tray 22 and a trial insert 24. The trial insert 24 may be temporarily attached to the tibial tray 22, or alternatively, may be integrally formed to provide a trial bearing surface. Trial inserts 24 may be manufactured to different shape and size specifications.

The materials used in a joint replacement surgery are designed to enable the joint to mimic the behavior or a normal knee joint. While various designs may be employed, in one embodiment, the femoral component 18 may comprise a metal piece that is shaped similar to the end of a femur 12, i.e., having groove 25 and condyles 26. The condyles 26 are disposed in close proximity to a bearing surface of the trial insert 24, and preferably fit closely into corresponding concave surfaces of the trial insert 24. The femoral and tibial components 18, 20 may comprise several metals, including stainless steel, alloys of cobalt and chrome, titanium, or another suitable material. Plastic bone cement may be used to anchor permanent prosthetic components into the femur 12 and tibia 16. Alternatively, the prosthetic components may be implanted without cement when the prosthesis and the bones are designed to fit and lock together directly, e.g., by employing a fine mesh of holes on the surface that allows the femur 12 and tibia 16 to grow into the mesh to secure the prosthetic components to the bone.

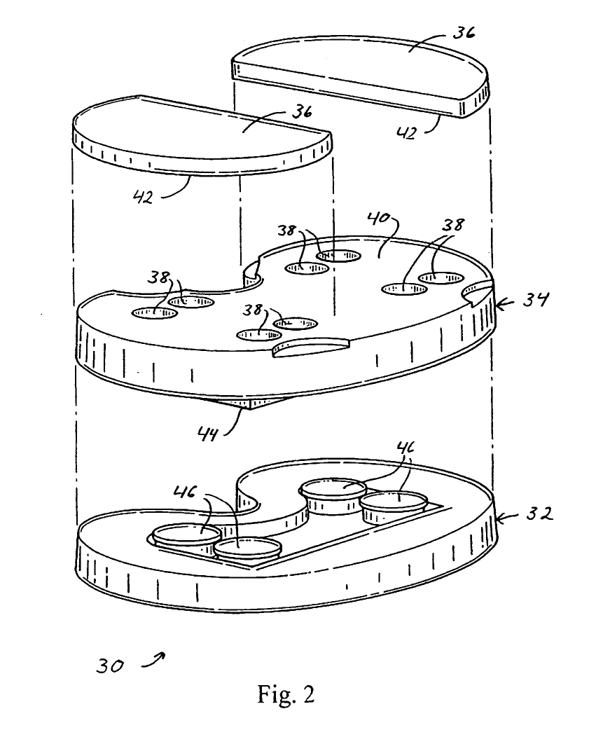

During the surgical procedure, prior to insertion of the femoral and tibial components 18, 20, and the trial insert 24, a spacer block is inserted within the knee 10 to gather data and assist the surgeon in determining whether additional bone must be removed and in selecting the appropriate trial insert 24. Referring to FIG. 2, an exploded view of a spacer block is shown generally at 30. The spacer block 30 includes a first body piece 32, a second body piece 34 positioned on top of the first body piece 32, and at least one chim 36 positioned on top of the second body piece 34.

As shown, for a knee replacement surgery, two chims 36 are mounted on top of the second body piece 34. The chims 36 are removably mounted onto the second body piece 34 to allow easy replacement of the chims 36. The chims 36 come in various thickness, and through trial and error, chims 36 having the proper thickness can be inserted to insure that the data collected by the spacer block 30 is accurate. As shown, the second body piece 34 includes recesses 38 formed in a top surface 40 thereof. The chims 36 have corresponding projections (not shown) extending from a bottom surface 42 thereof, that engage the recesses 38 of the second body piece 34 to secure the chims 36 thereon.

The first body piece 32 includes at least one sensor to measure forces between the upper and first body pieces 32, 34. A processor 44 having a memory is mounted within the second body piece 34 and is operatively connected to the sensors when the upper and first body pieces 32, 34 are assembled.

Referring to FIG. 2, a plurality of load cells 46 are positioned within the first body piece to measure compression, tension, and bending forces between the upper and first body pieces 34, 32. The load cells are operatively connected to the processor 44 so information related to the forces between the upper and first body pieces 34, 32 can be sent to the processor. At least one load cell 46 is associated with each chim 36.

As shown, the first body piece 32 includes two loads cells 46 for each chim 36. The load cells 46 are positioned immediately below the chims 36 such that the load cells 46 will measure forces between the upper and first body pieces 34, 32 due to forces exerted on the chim 36 positioned immediately above. More loads cells 46 will allow more information to be gathered regarding the forces on the chims 36. Ultimately, the appropriate number of load cells 46 used depends on the particular application.

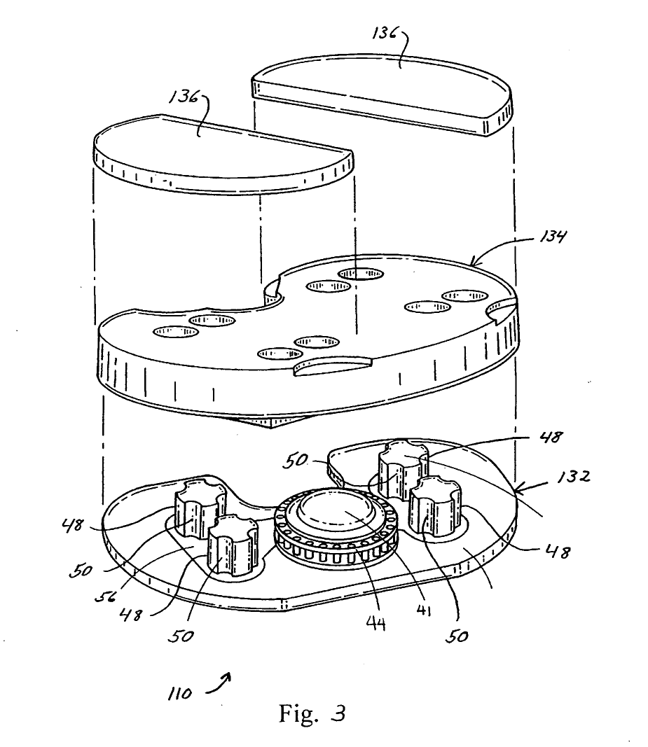

Referring to FIG. 3, another embodiment of the spacer block is shown generally at 110. This spacer block 130 includes chims 136, a second body piece 134, and a first body piece 132 similar to those described above. In the embodiment shown in FIG. 3, the first body piece 132 includes a plurality of poles 48 extending vertically upward in relation to first body piece 132.

Referring to FIG. 4, the second body piece 134 includes a plurality of pockets 49 formed therein. The pockets are sized to accommodate the poles 48 from the first body piece 132. When assembled, the poles 48 will be positioned in contact with the second body piece 134 within the pockets 49. There is no pre-load between the second body piece 134 and the poles 48, but any deflection of the second body piece 134 will cause the second body piece 134 to push against, and cause deflection of the poles 48.

The poles 48 have flat surfaces 50 formed on the sides. Alternatively, grooves or slots could also be formed within the sides of the poles 48. A plurality of strain gauges 52 are positioned on the flat surfaces 50 of the poles 48 to measure compression, tension, and bending forces experienced by the poles 48 due to contact from the second body piece 134.

The size of the pockets 49 formed in the second body piece 134 is precisely calibrated to allow deflection of the poles 48 and to insure that when the second body piece 134 and the first body piece 132 are assembled, and the poles 48 are inserted within the pockets 49, the strain gauges 52 are not damaged. The flat sides 50, grooves, or slots formed on the poles 48 provide a flat surface onto which the strain gauges 52 can be mounted, and provide a recessed area to protect the strain gauges from damage.

The second body piece 134 further includes a larger pocket 54 formed to accommodate a processor 144. The strain gauges 52 are operatively connected to the processor 144 via a printed circuit board or signal medium 56 so information related to the forces on the second body piece 134 can be sent to the processor 144. At least one pole 48 is associated with each chim 136.

As shown, the first body piece 132 includes two poles 48 for each chim 136. The poles 48 are positioned immediately below the chims 136 such that the strain gauges 52 will measure forces exerted on the chim 136 positioned immediately above. Referring to FIG. 3A, the strain gauges 52 are positioned at different orientations to allow the strain gauges 52 to gather force information along different directions. More strain gauges 52 will allow more information to be gathered regarding the forces on the chims 136. Ultimately, the appropriate number of poles 48 and strain gauges 52 used depends on the particular application.

It is to be understood that the sensors could be any appropriate sensing device. Strain gauges 52 and load cells 46 are cited herein as examples only, and the invention is not meant to be limited to these specific examples. Further, while the illustrative embodiments having four load cells 46 or four poles 48 and strain gages 52 is depicted in FIGS. 2 and 3, various other sensor configurations may be employed. For example, a sensor arrangement as described in applicant's co-pending U.S. Patent Application Pub. No. 2004/0019382 A1 may be employed. Specifics regarding the electronics involved in the present invention are described in applicant's co-pending U.S. patent application Ser. No. ______ (Attorney Docket No. 12462/6), filed concurrently herewith and entitled “Force Monitoring System.”

In the embodiment shown, a transmitter (not shown) is mounted within the processor 44, 144. The transmitter is adapted to take the data collected from the sensors 46, 52 by the processor 44, 144 and send the data to a remote receiver. Preferably, the receiver will analyze the data and provide feedback to help determine the proper sizing of the trial insert 24, as more fully discussed below. Processor 44, 144 may be powered by battery 41.

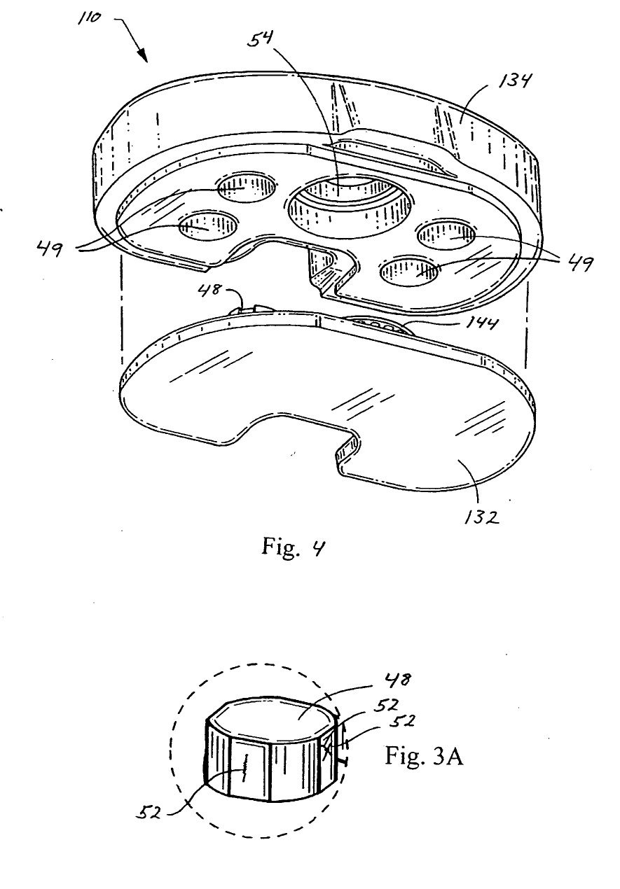

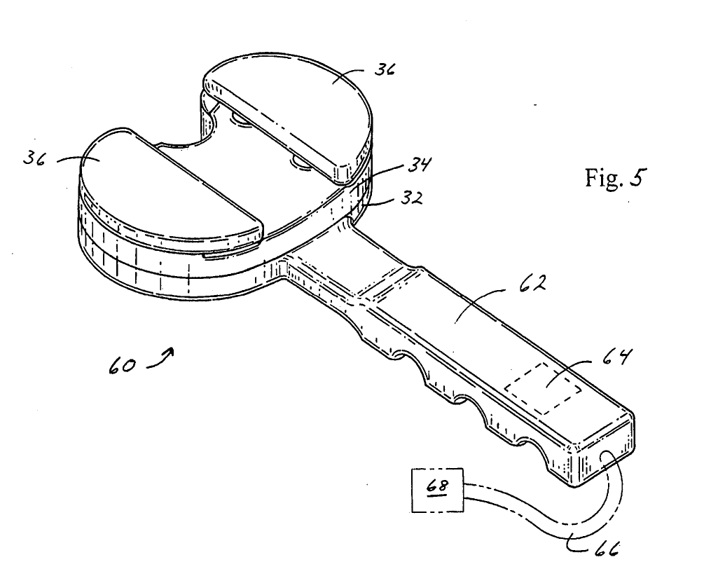

Referring to FIG. 5, a spacer block 60 having a handle 62 is shown. The handle 62 allows for easier manipulation and handling of the spacer block 60. The handle 62 of the spacer block 60 shown in FIG. 5 is integrally formed with the spacer block 60. The handle 62 includes a transmitter 64 operatively connected to the processor. The transmitter 64 is adapted to transmit data from the processor to a remote receiver. Alternatively, the handle 62 may include a hard wired connection 66 to a receiver 68 such that data from the processor can be sent to the receiver 68, through the handle 62, as shown in phantom in FIG. 5.

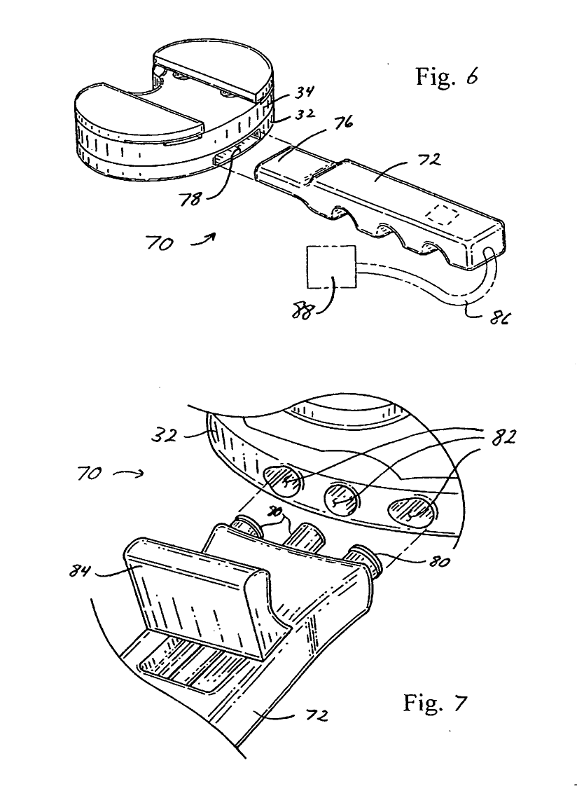

Referring to FIG. 6, a spacer block 70 is shown having a detachably mounted handle 72. The handle 72 and the spacer block 70 include features to allow an electrical connection therebetween when the handle 72 is connected to the spacer block 70. Any known electrical connector that is suitable for this particular application. One such electrical connection is shown in FIG. 6, wherein the handle 72 includes an insert portion 76, and the spacer block 70 includes a slot 78. The insert portion 76 and the slot 78 have electrical connectors that are brought into contact with one another when the insert portion 76 is inserted within the slot 78. This type of connection is well known, and is similar to the connection of a power cable to a cell phone or the like. This type of connection could also include threaded fasteners (not shown) to allow the handle 72 to be secured to the spacer block 70 after the insert portion 76 has been inserted within the slot 78.

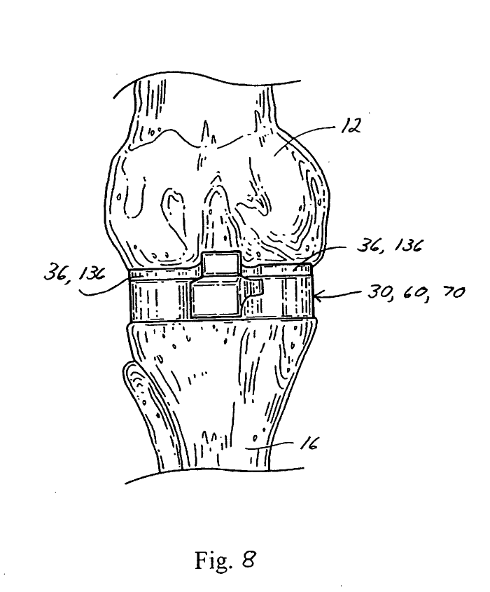

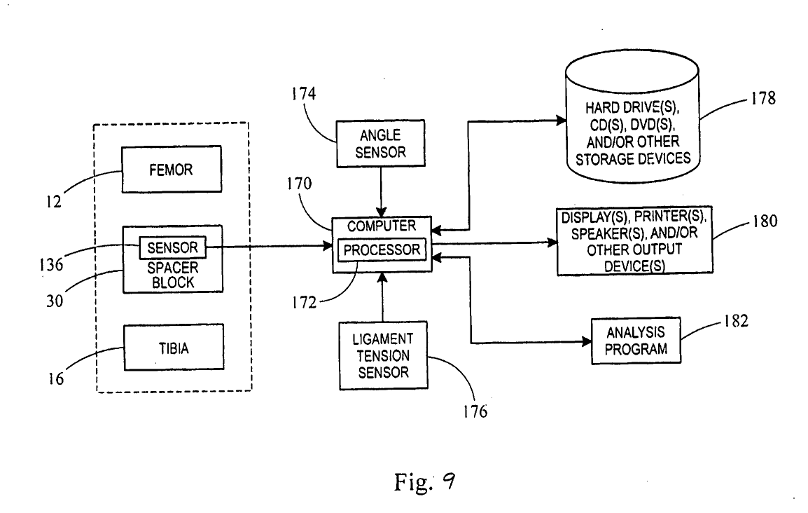

Further, another type of electrical connection is shown in FIG. 7, wherein the handle 72 includes projecting conductors 80 and the spacer block 70 includes openings 82 to receive the conductors 80. The conductors 80 may be asymmetrical and rotatable, such that after insertion into corresponding shaped openings 82, the conductors 80 may be rotated by actuating a lever 84, thereby locking the handle 72 to the spacer block 70.

As described above, the detachable handle 72 may also include a transmitter 74 that is operatively connected to the processor through the electrical connection between the handle 72 and the spacer block 70. The transmitter 74 is adapted to transmit data from the processor to a remote receiver, when the handle 72 is connected to the spacer block 70. Alternatively, the handle 72 may include a hard wired connection 86 to a receiver 88 such that data from the processor can be sent to the receiver 88, through the handle 72, when the handle 72 is connected to the spacer block 70, as shown in phantom in FIG. 6.

Referring to FIG. 8, when the spacer block 30, 60, 70 is fully assembled and disposed between the femur 12 and the tibia 16, the sensors (strain gauges 52, or load cells 46) are responsive to the forces imposed by the femur 12 upon the chims 36, 136. Furthermore, the sensors may provide data in a real-time, or near real-time fashion, allowing for intraoperative analysis of the data. Specifically, the processor 44, 144 contains a memory for storing the data. In operation, the processor 44, 144 is adapted to receive, as an input, multiple sensor outputs created by each of the strain gages 52 or load cells 46 in response to forces exerted on the chims 36, 136. The processor 44, 144 may be coupled to a transmitter 64, 74 that is adapted to convert the multiple sensor inputs to a data stream, such as a serial data stream, and transmit the data stream, via wired or wireless connection, to a receiver 68, 88 as described above.

As shown in FIG. 9, a computer 170 having processor 172 and a memory coupled thereto is in communication with at least one sensor 136, which is embedded within the spacer block 30. If desired, the computer 170 may communicate with ancillary components 178, 180, and 182, as described in greater detail in applicant's co-pending U.S. Patent Application Pub. No. 2004/0019382 A1. For example, in one embodiment described in greater detail below, the output device 180 may display neural network data in terms of a force and position of the force imposed upon a joint. Further, if desired, optional joint angle sensor 174 and optional ligament tension sensor 176 may be used during the joint replacement procedure to acquire additional data, as generally described in applicant's above-referenced application.

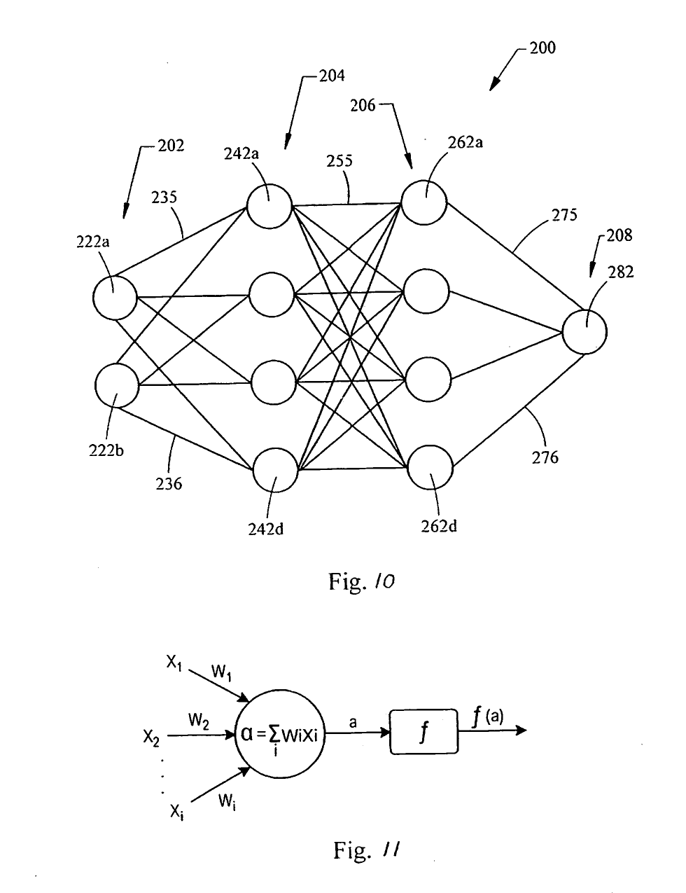

Referring now to FIGS. 10 and 11, an introduction to neural networking principles is provided. Data from the sensors in the spacer block 30 will be analyzed in this manner and as described in applicant's co-pending U.S. patent application Ser. No. ______ (Attorney Docket No. 12462/4), filed concurrently herewith and entitled “Application of Neural Networks to Prosthesis Fitting and Balancing in Joints.”

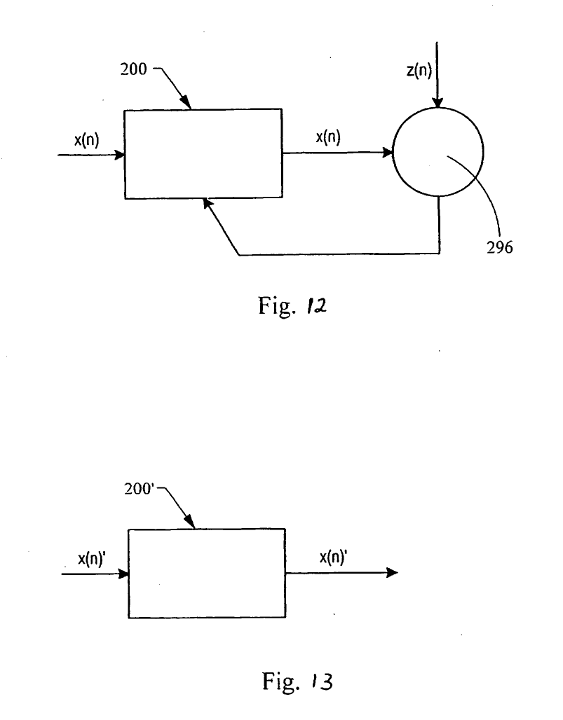

As will be described in greater detail below with respect to FIGS. 12 and 13, the neural networking principles may be used in conjunction with a joint replacement procedure to provide improved data acquisition ability and simplify the procedure. For example, known force and position data acquired by sensors of a spacer block 30 may be passed through a trained neural network, which can predict and output at least one previously unknown force and location. The outputted, predicted data values may be made available to a physician and used, for example, to aid in the determination of whether to resect additional bone, release soft tissues, and/or select sizes for the trial insert during the joint replacement procedure.

In FIG. 10, a basic overview of one exemplary neural network is shown. Neural network 200 generally encompasses analytical models that are capable of predicting new variables, based on at least one known variable. The neural network comprises a specific number of “layers,” wherein each layer comprises a certain number of “neurons” or “nodes.” In the embodiment of FIG. 10, neural network 200 comprises input layer 202, first layer 204, second layer 206, and output layer 208. First and second layers 204 and 206 are commonly referred to as “hidden layers.”

In the embodiment of FIG. 10, exemplary input parameters 222a and 222b are provided. While two input parameters are shown for simplicity, it is preferred that as many input parameters as possible are included to achieve improved prediction accuracy. In the context of total joint replacement, various input parameters may be employed. The inputs may comprise “static” variables, such as the age, height, weight and other characteristics of the patient. The inputs may also comprise “dynamic” variables, such as data acquired by sensors of the spacer block 30, 60, 70. In practice, virtually any combination of static and dynamic variables may be inputted into the neural network. The aggregate input is generally represented by input layer 202.

A plurality of “connections,” which are analogous to synapses in the human brain, are employed to couple the input parameters of input layer 202 with the nodes of first layer 204. In the embodiment of FIG. 10, illustrative connection 235 couples input parameter 222a to first layer node 242a, while connection 236 couples input parameter 222b to node 242d. A different connection is employed to couple each input parameter to each node of the first layer. In FIG. 10, since there are two input parameters and four nodes in first layer 204, then eight connections total are employed between input layer 202 and first layer 204 (for simplicity, only connections 235 and 236 have been numbered). However, as noted above, any number of input parameters may be employed, and any number of first layer nodes may be selected. Therefore, the number of connections may vary widely. Moreover, as explained below, each connection has a weighted value associated therewith.

Each node in FIG. 10, is a simplified model of a neuron and transforms its input information into an output response. FIG. 10 illustrates the basic features associated with input, weighting, activation and transformation of a single node. In a first step, multiple inputs x₁-xᵢ are provided to each node. Each input x₁-xᵢ has a weighted connection w₁-wᵢ associated therewith. The activation “a” of a node is computed as the weighted sum of its inputs, as shown in FIG. 11. Finally, a transfer function “f” is applied to the activation value “a” to obtain output value “f(a)”, as shown in FIG. 11. The output value “f(a)” of a particular node then is propagated to the node of a subsequent layer for further processing.

Transfer function “f” may encompass any function whose domain comprises real numbers. While various transfer functions may be utilized, in one embodiment, a hyperbolic tangent sigmoidal function is employed for nodes within first hidden layer 204 and second hidden layer 206, and a linear transfer function is used for output layer 208. Alternatively, a step function, logistic function, and normal or Gaussian function may be employed.

In sum, any number of hidden layers may be employed between input layer 202 and output layer 208, and each hidden layer may have a variable number of nodes. Moreover, a variety of transfer functions may be used for each particular node within the neural network.

Since neural networks learn by example, many neural networks have some form of learning algorithm, whereby the weight of each connection is adjusted according to the input patterns that it is presented with. Therefore, before neural network 200 may be used to predict unknown parameters, such as contact locations and forces that may be experienced in the context of total joint replacement surgery, it is necessary to “train” neural network 200.

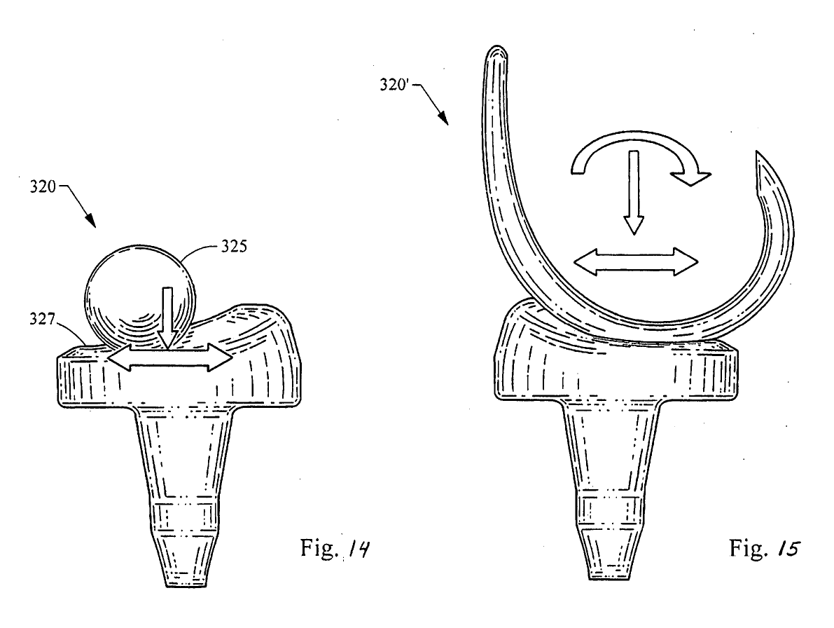

In order to effectively train neural network 200, it is important to have a substantial amount of known data stored in a database. The database may comprise information regarding known contact forces and their locations. Data samples may be acquired using various techniques. For example, as described with respect to FIGS. 14, and 15 below, known position and load values may be obtained using computer analysis models, such as finite element modeling. Alternatively, sample data values may be obtained using a load testing machine, such as those manufactured by Instron Corporation of Norwood, Mass. The sample data values representative of position and load may be stored in processor 172 of computer 170.

The data samples may be separated into three groups: a training set, a validation set, and a test set. The first set of known data samples may be used to train neural network 200, as described below with respect to FIG. 12. The second set of known data samples may be used for validation purposes, i.e., to implement early stop and reduce over-fitting of data, as described below. Finally, the third set of known data samples may be used to provide an error analysis on predicted sample values.

Referring now to FIG. 12, a block diagram showing the training phase of neural network 200, for use in conjunction with prosthesis fitting and balancing in joints, is described. A key feature of neural network 200 is that it may learn an input/output relationship through training. Neural network 200 may be trained using a supervised learning algorithm, as described below, to adjust the weight of the connections to reduce the error in predictions. The training data set may be used to train the neural network using MATLAB or another suitable program. In the context of a joint replacement procedure, neural network 200 may take one or more input parameters, e.g., sensor values obtained from sensor 136, and predict as output one or more unknown parameters, e.g., contact positions and loads that ultimately may be imposed upon a permanent component.

In a first training step, an input value “x(n)” is inputted into neural network 200. After being processed through neural network 200, a predicted output value, generally designated “y(n),” is obtained. It should be noted that predicted output value y(n) of FIG. 12 is the same value as output 282 of FIG. 10. Predicted output y(n) then is compared to a target value, generally designated “z(n).” Error logic 296, such as a scalar adder logic, then compares predicted output value y(n) with target value z(n).

In the context of joint replacement surgery, input value x(n) may comprise measured sensor values indicative of position and load. Further, target value z(n) may comprise known sample data representative of position and load. The known sensor values x(n) are fed through neural network 200 and predicted output y(n) is obtained. Logic 296 compares the estimated output y(n) with known target value z(n), and the weight of the connections are adjusted accordingly.

The supervised learning algorithm used to train neural network 200 may be the known Bayesian Regularization algorithm with early stopping. Alternatively, neural network 200 may learn using the Levenberg-Marquardt learning algorithm technique with early stopping, either alone or in combination with the Bayesian Regularization algorithm. Neural network 200 also may be trained using simple error back-propagation techniques, also referred to as the Widrow-Hoff learning rule.

As noted above, a set of data samples may be used for validation purposes, i.e., to implement early stop and reduce over-fitting of data. Specifically, the validation data samples may be used to determine when to stop training the neural network so that the network accurately fits data without overfitting based on noise. In general, a larger number of nodes in hidden layers 204 and 206 may produce overfitting.

Finally, a third set of known data samples may be used to provide an error analysis on predicted sample values. In other words, to verify the performance of the final model, the model is tested with the third data set to ensure that the results of the selection and training set are accurate.

Referring now to FIG. 13, a use phase of neural network 200 is shown. The use phase may be employed to predict contact forces during a joint arthroplasty procedure. Contact forces that may be experienced during or after surgery may be estimated. During surgery, only a limited number of sensors are disposed within the spacer block 30, 60, 70. Instead of yielding data representative of only those sensors, neural network 200 may use the limited data from sensors to predict position and load values for numerous other locations. Advantageously, the enhanced feedback provided to the physician may be used to aid in balancing soft tissue during the arthroplasty procedure.

In FIG. 13, sensor value x(n)′ is fed through previously-trained neural network 200′ to obtain at least one previously unknown data value y(n)′. Sensor value x(n)′ may comprise data representative of load and position, as measured by the sensors. As noted above, sensors may intraoperatively collect data representative of a force imposed on the spacer plates during flexion or extension of the knee. During the medical procedure, the physician may maneuver the knee joint so that sensors collect real-time data. This sensor data x(n)′ may be operatively coupled to processor 172, so that processor 172 may implement the trained neural network algorithms to predict unknown data values.

Advantageously, by employing neural network techniques in conjunction with data sensing techniques of the present invention, a physician may obtain significant amounts of estimated data from only a few data samples. During a prosthesis fitting procedure, the physician only needs to insert one spacer block 30, 60, 70 having sensors 48, 52 embedded therein. The physician need not “try out” multiple spacer blocks 30, 60, 70 to determine which trial insert 24 is an appropriate fit before implanting permanent components. Rather, by employing the neural networking techniques described herein, the physician may employ one spacer block 30, 60, 70, acquire a limited amount of force/position data, and be provided with vast amounts of data to aid in the determination of whether to resect additional bone, release soft tissues, and/or select sizes for the trial insert during the joint replacement procedure.

Further, by employing the neural networking techniques described herein, the physician need not substantially rely on verbal feedback from a patient during a procedure. By contrast, the physician may rely on the extensive data provided by the neural network software, thereby facilitating selection of permanent prosthetic components. Moreover, it is expected that the prosthetic components will experience reduced wear post-surgery because of improved component selection and/or the ability to properly balance soft tissue during surgery based on the neural network data available to the physician.

Another advantage of using the neural network technique of the present invention in a joint replacement procedure is that the database of stored values can grow over time. For example, even after a neural network is trained and used in procedures to predict values, sensed data may be inputted and stored in the database. As the database grows, it is expected that improved data estimations will be achieved.

As noted above, it will be appreciated that while the techniques of the present invention have been described in the context of acquiring data using a spacer block or trial insert during a knee replacement procedure, data also may be acquired and/or processed while a permanent component is housed within the patient. In the latter embodiment, the permanent component may utilize the apparatus and techniques described above to provide feedback to a physician while the component is housed within the patient's body, i.e., after surgery.

Referring now to FIGS. 14 and 15, methods for collecting data for use in creating a database of known solutions for training a neural network are provided. As noted above, in order to effectively train neural network 200, it is important to have a substantial amount of existing, known data stored in a database. In FIGS. 13 and 14, data samples indicative of position and load are obtained using finite element modeling. In FIG. 14, finite element model 320 is shown. A load, represented by sphere 325, is dragged over simulated bearing surface 327. The load preferably is cycled throughout bearing surface 327 in an anterior/posterior direction and a medial/lateral direction. The load imposed may range, for example, from about 0 to 400 N. Preferably, hundreds or thousands of sample data points are collected. At each load point, a sensor reading indicative of position and load is stored in the database of known solutions, e.g., in processor 172 of computer 170.

In FIG. 11, finite element model 320′ is similar to finite element model 320, with the main exception that joint flexion between 0-90 degrees is simulated. Optionally, internal rotation of the joint, e.g., between −10 to 10 degrees, may be simulated. For each simulated flexion and/or rotation condition, model 320′ imposes a load on the bearing surface to obtain numerous sample data points. The sample data is stored in the database of known solutions in processor 172 and may be used to train, validate and test neural network 200, as described above. The finite element data gathered from models 320 and 320′ may be used alone or in combination with sample data obtained using a load testing machine, such as those manufactured by Instron Corporation, as described above.

In alternative embodiments of the present invention, the outputs from sensors may be transmitted to processor 172, wherein they may be captured by an analysis program 182, as shown in FIG. 8. Analysis program 182 may be a finite element analysis (“FEA”) program, such as the ANSYS Finite Element Analysis software program marketed by ANSYS Inc., located in Canonsburg, Pa., and commercially available. The FEA analysis program may display the data in a variety of formats on display 180. In one embodiment, sensor measurements captured by the FEA analysis program are displayed as both a pressure distribution graph and as a pressure topography graph, as described in applicant's above-referenced, co-pending U.S. Patent Publication No. 2004/0019382 A1.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

1.-18. (canceled)

19. A system that gathers information to select a trial insert, comprising:
a first body piece and a second body piece positioned on top of the first body piece;
a sensor positioned between the first body piece and the second body piece;
a processor positioned between the first body piece and the second body piece, the processor coupled with the sensor; and
a chim removably mounted to an exterior surface of the second body piece, the chim positioned in relation to the sensor such that a force exerted on the chim is detected by the sensor.

20. The system of claim 19, where the first body piece comprises a pole extending vertically upward in relation to a first surface of the first body piece, the sensor mounted on the pole.

21. The system of claim 20, where the second body piece comprises an aperture configured to receive the pole.

22. The system of claim 20, where the sensor is configured to measure an amount of compression experienced by the pole as a result of a force exerted on the chim.

23. The system of claim 20, where the sensor is configured to measure an amount of tension experienced by the pole as a result of a force exerted on the chim.

24. The system of claim 20, where the sensor is configured to measure an amount of bending force experienced by the pole as a result of a force exerted on the chim.

25. The system of claim 19, where the sensor comprises a load cell.

26. The system of claim 19, where the sensor comprises a strain gauge.

27. The system of claim 19, further comprising a transmitter configured to transmit data from the processor to a remote receiver.

28. The system of claim 19, further comprising a handle mounted to the first or second body piece.

29. The system of claim 28, where the handle is detachably connected to the first or the second body piece.

30. The system of claim 28, where the handle and the first or the second body pieces comprise an electrical connection.

31. The system of claim 30, where the handle comprises a transmitted coupled with the processor through the electrical connection, the transmitter configured to transmit data between the processor and a remote processor.

32. A system that gathers information used to select a trial insert, comprising:
a first body piece comprising a plurality of poles extending vertically upward in relation to a first surface of the first body piece;
a second body piece configured to mate with the first body piece;
a chim removably mounted to an exterior surface of the second body piece; and
means for measuring a force experienced by at least one of the poles as a result of a force exerted on the chim.

33. The system of claim 32, further comprising means for transmitting a measured force to a remote processor.

34. A method to select a joint trial insert, comprising:
providing a spacer block comprising a first body piece and a second body piece mated together, the first body piece comprising a pole extending vertically upward from a first surface of the first body piece, and the second body piece comprising an aperture to receive the pole when the first and second body pieces are mated together;
mounting a chim to an exterior surface of the spacer block;
inserting the chim and spacer block into a joint;
manipulating the joint so a force is exerted on the chim; and
collecting data representative of a difference between an initial position of the pole and a later position of the pole resulting from the force exerted on the chim,
where at least one sensor is mounted on the pole.

35. The method of claim 34, further comprising analyzing the collected data to determine whether to mount a thicker or thinner chim on the spacer block.

36. The method of claim 35, further comprising mounting a different size chim on the spacer block.

37. The method of claim 35, where the act of analyzing the collected data comprises performing a neural network analysis on the collected data.

38. The method of claim 34, where the act of collecting data comprises transmitting data representative of the difference between an initial position of the pole and the later position of the pole resulting from the force exerted on the chim to a remote processor in substantially real-time.
